(19) United States
(12) Patent Application Publication
(10) Pub. No.: US 2025/0339626 A1
(43) Pub. Date: Nov. 6, 2025

(54) A METHOD OF ASSEMBLING A NEEDLE SAFETY APPARATUS

(71) Applicant: TIP-TOP.COM LTD, Colchester (GB)

(72) Inventor: Barry P. Liversidge, Colchester (GB)

(73) Assignee: TIP-TOP.COM LTD, Colchester (GB)

(21) Appl. No.: 18/867,067

(22) PCT Filed: May 19, 2023
(86) PCT No.: PCT/GB2023/051324

§ 371 Date: Nov. 19, 2024

(30) Foreign Application Priority Data
May 20, 2022 (GB) 2207437.1

(51) Int. Cl. A61M 5/32; A61M 5/00; B65B 5/04; B65B 7/16; B65B 25/00

(52) U.S. Cl. CPC A61M 5/3232; A61M 5/002; A61M 5/3257; A61M 5/3276; B65B 5/04; B65B 7/16; B65B 25/00; A61M 2207/00

(57) ABSTRACT

The present invention provides a method of assembling and packaging a medical needle device having a double ended needle (116), which comprises a container (170) having a closed end and an open end to receive a spring activated safety needle device (110) to provide passive needlestick protection to a non-patient end (118) of the needle (116). The method comprises performing the independent steps of (i) assembling a first sub-assembly (101) by mounting a shield (114) to a tubular housing (130) to form a telescopic device (104) and engaging the telescopic device (104) within the container (170), and (ii) assembling a second sub-assembly (102) by securing the double ended needle (116) within the needle mount (140), wherein the mounting element is provided on an external surface of the needle mount (140). The tubular housing (130) comprises (a) a mounting member on an internal surface to engage a mounting element of the needle mount (140), and (b) a spring to urge movement of the double ended needle (116) during use to a shielding position whereat the needle (116) extends at an angle oblique to a longitudinal axis of the tubular housing (130) to place the non-patient end (118) of the needle (116) at a location adjacent to an interior wall of the tubular housing (130). The method further comprises inserting the second sub-assembly (102) into the tubular housing (130) to secure the mounting element of the needle mount (140) to the mounting member of the tubular housing (130) to unite the second sub-assembly (102) to the first sub-assembly (101) to enable the spring to urge movement of the double ended needle (116) during use to a shielding position. Finally, the method comprises applying the closure a label (124) over the open end of the container (170) to seal the medical needle device 110 within the container (170).

### FIELD OF THE INVENTION

The present invention relates to a method of assembling a safety needle device (assembly) and an assembly line process for assembling a safety needle device. In particular, the present invention relates to a method of assembling a safety needle device which provides a shielding arrangement to confer automatic (passive) needlestick protection and, specifically, provides a pivotally/tilt mounted needle to prevent needlestick injuries at the non-patient end of the needle after use.

### BACKGROUND TO THE INVENTION

Needlestick injuries may be caused by the distal tip of the needle which is used to penetrate through the skin. However, the proximal end of the needle is also sharp since it is used to pierce an elastomeric membrane and project into a cartridge or container holding a liquid drug. Once the safety needle device (assembly) is removed from the injecting device, the proximal non-patient end of the needle therefore presents a further risk to a clinician or user. Accordingly, the distal end of the safety needle assembly may include a shielding device for the distal tip of the needle and may also comprise a shroud or skirt which affords some protection to/of the proximal end tip of the needle. Furthermore, the needle may be incorporated in a needle safety device such that the needle pivots/tilts as the safety needle device is detached from the medical injector. In such a device, the proximal tip of the needle locates adjacent to or abuts a shroud or skirt such that a fingertip cannot easily make contact with the proximal end of the needle tip.

In order to provide such needlestick safety protection as described above, the safety needle device requires a metal spring or a number of metal springs to actuate the shielding arrangement and/or to pivot/tilt the needle. These springs may need to be compressively preloaded and or also tensioned torsionally as the safety needle device is being assembled. Clearly, the positioning and tensioning of such springs within these small safety devices is very intricate and care must be taken not to damage either the distal or proximal ends of the needle within such devices. Accordingly, the use of metal springs is problematic and significantly increases the cost and also decreases the pen needle assembly line speed. Such safety needle devices are typically mass produced at very high speed.

Furthermore, needle safety devices must be quality checked to a significant degree due to the eventual use of the devices. Accordingly, assembly methods and in particular pen needle assembly lines utilise numerous quality checks including video inspection at each individual assembly stage. The greater the number of assembly steps the greater the number of quality checks need to be completed which further increases assembly cost and decreases the speed of the assembly line.

The term “use” or “during use” are used to cover the complete use of the safety needle device, for example from the act of attaching a medical injector to the safety needle device and removing it from the container, then performing the injection on a patient, and then detaching the safety needle device from the medical injector and finally disposing of the safety needle device into a sharps waste container.

It is an aim of the present invention to overcome at least one problem associated with the prior art, whether referred to herein or otherwise.

### SUMMARY OF THE INVENTION

According to a first aspect of the present invention there is provided a method of assembling and packaging a medical needle device having a double ended needle, which comprises a container having a closed end and an open end to receive a spring activated safety needle device to provide passive needlestick protection to a non-patient end of the needle; and wherein the open end of the container receives a closure to maintain sterility within the container, the method comprising:
performing the independent steps of:
   (i) assembling a first sub-assembly comprising a telescopic device engaged within the container wherein the telescopic device comprises a shield and a tubular housing, and wherein the tubular housing comprises:
      (a) a mounting member on an internal surface to engage a mounting element of a needle mount; and
      (b) a spring means to urge movement of the double ended needle during use to a shielding position whereat the needle extends at an angle oblique to a longitudinal axis of the tubular housing to place the non-patient end of the needle at a location adjacent to an interior wall of the tubular housing;
   (ii) assembling a second sub-assembly by securing the double ended needle within the needle mount, wherein the mounting element is provided on an external surface of the needle mount;
inserting the second sub-assembly into the tubular housing to secure the mounting element of the needle mount to the mounting member of the tubular housing to unite the second sub-assembly to the first sub-assembly to enable the spring means to urge movement of the double ended needle during use to a shielding position;
applying the closure over the open end of the container to seal the medical needle device within the container.

The assembling of the first sub-assembly may comprise securing a part of the telescopic device within the open-ended container using an interference fit. The assembling of the first sub-assembly may comprise securing a part of the shield in the open-ended container using an interference fit. The assembling of the first sub-assembly may comprise securing a part of the tubular housing in the open-ended container using an interference fit.

The interference fit to releasably retain the safety needle device within the open-ended container may comprise detents or cam profiles formed within the container to interact with a part of the telescopic device.

Preferably the assembling of the first sub-assembly is undertaken on a first assembly line and the assembling of the second sub-assembly is undertaken on a second assembly line. Preferably the first sub-assembly is united/coupled to the second sub-assembly on the second assembly line. Preferably the first assembly line is separate and/or distinct from the second assembly line. The first and second assembly lines may comprise parallel assembly lines. The first assembly line may be remotely located from the second sub-assembly line. The method may comprise storing the first sub-assemblies for a period of time prior to uniting/coupling the first sub-assemblies with the second sub-assemblies. The present invention thereby addresses a problem introduced by the “Use By” shelf life of such products which commences on securement of the sterile barrier label over the open end of the container. For example, the completion of the first sub-assemblies does not trigger the beginning of this shelf-life period. Such first assemblies can be assembled and stored at any location for a significant period of time prior to being used. This provides the opportunity of the first sub-assemblies being manufactured in a country where the manufacture and storage may be low cost. In addition, the final manufacturer can be assured that they have the required number of complete first sub-assemblies prior to running the second sub-assembly line. In addition, the speed of the second assembly line is not influenced in anyway (i.e. slowed down) by assembling the first sub-assemblies along with the uniting of the first sub-assemblies with the second sub-assemblies. The speed of the assembly line is therefore influenced by the speed of assembly of the second sub-assembly and/or the speed of uniting the first sub-assembly with the second sub-assembly.

The method may comprise transporting the first sub-assemblies from the first assembly line to the second assembly line.

The method may comprise packing a plurality of first sub-assemblies into bulk packaging, transporting the bulk packaged items to the second assembly line, removing the first sub-assemblies from the bulk package and introducing individually each first sub-assemblies into the second assembly line.

The method may comprise push-fitting the second sub-assembly into the first sub-assembly in order to unite the first and second sub-assemblies.

The uniting of the first sub-assembly with second sub-assembly may comprise moving the first sub-assembly along a central longitudinal axis relatively towards the second sub-assembly and the uniting may comprise this single linear movement.

Preferably the method comprises aligning the first sub-assembly with the second sub-assembly. Preferably this alignment only requires an alignment of central longitudinal axes of the first and second sub-assemblies and does not necessitate (omits) any relative rotational radial alignment of the sub-assemblies about the respective central longitudinal axis to unite the two sub-assemblies together.

Preferably the method comprises aligning a central longitudinal axis of the first sub-assembly with a central longitudinal axis of the second sub-assembly and then solely moving the first sub-assembly along the coincident central longitudinal axes relatively towards the second sub-assembly.

Preferably the needle mount is rotatable about a central longitudinal axis in order for the needle mount to remain engageable within the mounting member at any relative rotational position.

Preferably the mounting element is engageable within the mounting member at any relative rotational position relative to a central longitudinal axis of the needle mount (and/or the second sub-assembly) and/or the tubular housing (and/or first sub-assembly).

The needle mount may comprise a longitudinal axis in which the needle is arranged to extend along the longitudinal axis, wherein the needle mount is rotatable about the longitudinal axis whereby the mounting element remains engageable within the mounting member to articulate about any angular position/orientation around the longitudinal axis.

Preferably the method comprises tiltably mounting the needle mount within the tubular housing as the first sub-assembly is united with the second sub-assembly.

Preferably the method comprises maintaining the spring in an unloaded configuration as the first sub-assembly is united with the second sub-assembly. Preferably the spring is preloaded when the medical injector (pen injector) is attached to the safety needle device.

The assembling of the first sub-assembly may comprise securing the tubular housing in the open-ended container using an interference fit.

The assembling of the first sub-assembly may comprise (in the first assembly line) forming the telescopic device and then removably inserting the telescopic device into the container to form the first sub-assembly. The method may comprise push fitting the telescopic device into the container. The method may comprise push fitting the shield into the container. The method may comprise push fitting the tubular housing into the container.

The method may comprise push fitting the shield over the tubular housing. The method may comprise push fitting the shield within the tubular housing.

The uniting of the first sub-assembly with the second sub-assembly comprises push fitting the mounting element into the mounting member.

The method may comprise inserting the second sub-assembly into the first sub-assembly with the sub-assemblies being in any relative rotational orientation about a central longitudinal axis. The central longitudinal axis may be defined by the central longitudinal axis of the first sub-assembly and the central longitudinal axis of the second sub-assembly and the method may comprise moving these longitudinal axes so as to be coincident.

The mounting element may comprise a shaped outer surface of the needle mount. The mounting element may comprise a ball-shaped outer surface of the needle mount which may facilitate a “ball and socket joint” together with the mounting member. The shaped outer surface may comprise a curved outer surface (or any other surface/shape so as to allow pivoting articulated connection between the first and second sub-assembly). The shaped outer surface may comprise a partial spherical surface. The shaped outer surface may be uniform around a central longitudinal axis of the needle mount.

The method may comprise locating the mounting element within the mounting member. The method may comprise pushing the mounting element into the mounting member and may comprise pushing the shaped outer surface of the mounting element to locate within the shaped inner surface of the mounting member such that the mounting element may articulate (and may move within) the mounting member whist the mounting element is preferably retained within the inner shaped surface of the mounting member.

The method may comprise forming a connection between the mounting element and the mounting member to permit articulation, and/or limiting relative longitudinal movement between the 1^{st} (first) and 2^{nd} (second) sub-assemblies.

The method may comprise locating first and second mounting elements within first and second mounting members respectively. The first and second mounting elements may comprise stub axles (and/or axle lugs). The method may comprise mounting the needle mount between a pair of mounting members. The mounting members may comprise recesses. The method may comprise mounting the needle mount to form a tiltable arrangement as shown in GB2104342.7, GB 2108399.3 and GB 2201934.3.

The method may comprise mounting the needle mount between a first pair of mounting members and providing a second pair of mounting members whereby the needle mount is mounted to be moveable from the first pair of mounting members to the second pair of mounting members during use.

The method may comprise mounting the needle mount about an axis defined by the mounting element(s) of the needle mount.

Preferably the method comprises mounting the needle in an operative position, and mounting the spring and the needle mount at a set position, thereafter use of the spring activated safety needle device with a medical injector causes a shift from the set position, said shift enables the spring to rotate the needle to the shielding position when the spring activated safety needle device is detached from the medical injector. The method may comprise mounting the needle mount to form a tiltable arrangement as shown in GB 2108399.3 and GB 2201934.3.

Preferably the spring means comprises a resilient member which extends inwardly from the tubular housing and contacts an outer surface of the needle mount at a position offset from the axis of rotation and preferably applies a rotational force to rotate the needle mount.

Preferably the spring comprises a leaf spring projecting inwardly from the tubular housing and, with the needle in an operative position, the leaf spring is in a preloaded condition so as to be deflected from a neutral/relaxed position. Preferably with the needle in the operative position the leaf spring has energy stored therein. An end surface of the leaf spring may contact an outer surface of the needle mount to create a torque about an axis of rotation.

Preferably the needle mount comprises a unitary component having an integral mounting element or mounting elements in the form of a shaped surface or axial members located on an outer surface. The shaped outer surface may provide spherical surface and may enable the needle mount to be secured by a ball and socket arrangement and may enable a click-fit (ball and socket) arrangement. The axial members may provide hemispherical surfaces and may enable the needle mount to be secured by ball and socket arrangements and may enable a “click-fit” (ball and socket) arrangement.

The tubular housing may comprise mounting members in the form of axial members located on an internal surface for cooperation with the axial members provided on the needle mount. Preferably the axial members of the needle mount and the tubular housing enable a push fit engagement of the needle mount into the pivoting position within the axial members of the tubular housing. The method may comprise mounting the needle mount to form a tiltable arrangement as shown in GB2104342.7, GB 2108399.3 and GB 2201934.3.

The axial members of the tubular housing and the needle mount may comprise a first pair of axial members comprising projecting portions and a second pair of axial members comprising corresponding recesses. The projecting portions may comprise hemi-spherical projections. The axial members may provide a ball and socket joint. Preferably the axial members provide a first ball and socket joint on one side of the needle mount and a second ball and socket joint on an opposite side of the needle mount for connection to complementary ball and socket joints provided within the tubular housing.

The open (proximal) end of the container may comprise the proximal end and the closed end may comprise a distal end of the container.

### BRIEF DESCRIPTION OF THE DRAWINGS

The present invention will now be described, by way of example only, with reference to the drawings that follow, in which:

FIG. 1 is a schematic representation of an assembly line of a prior art pen needle device;

FIGS. 2A and 2B are a side view and a perspective view of a pen needle comprising a spring activated safety needle device in a configuration before an injection;

FIGS. 3A and 3B are a side view and a perspective view of a pen needle comprising a spring activated safety needle device in a configuration during an injection;

FIGS. 4A and 4B are a side view and a perspective view of a pen needle comprising a spring activated safety needle device in a configuration after an injection;

FIG. 5 is a perspective view of a pen needle comprising a detached spring activated safety needle device with the non-patient end of the needle in a shielded position;

FIG. 6 is an exploded view of a spring activated safety needle device and an assembly fixture;

FIGS. 7A and 7B are a side view and a perspective view of a needle and needle mount located on an assembly fixture with an open-ended container and spring activated sub-assembly in a pre-assembled position;

FIGS. 8A and 8B are a side view and a perspective view of a spring activated safety needle device in an assembled position within an open-ended container and located on an assembly fixture;

FIG. 9 is a partial cut away view of a preferred embodiment of a spring activated safety needle device prior to the coupling/uniting of the first sub-assembly with the second sub-assembly;

FIG. 10 is a partial cut away view of a preferred embodiment of a spring activated safety needle device showing the first sub-assembly coupled/united to the second sub-assembly;

FIG. 11 is a partial cut away view of a preferred embodiment of a spring activated safety needle device attached to a medical injector prior to an injection;

FIG. 12 is a partial cut away view of a preferred embodiment of a spring activated safety needle device detached from a medical injector in a used configuration; and

FIG. 13 is a schematic representation of a preferred embodiment of an assembly line of a spring activated safety needle device.

### DESCRIPTION OF THE PREFERRED EMBODIMENTS

The assembly method and assembly line(s) of the present invention is primarily but not exclusively intended for use with a medical needle which provides front end and back end needlestick protection. The medical needle may be used to penetrate a human or animal body, or may be used for other medical uses such as the penetration of a pierceable membrane of an intravenous medication system.

In the following description all uses of the needle safety assembly will be described simply as the penetration of a body, even though specific embodiments may be intended for other uses.

Throughout this specification and with reference to the figures, a safety needle device is shown and described herein which provides for shielding of a needle on a pen needle assembly specifically on the non-patient (proximal) end but also on the patient (distal) end. As used herein, the term “distal” and/or “forwards” or “forwardly”, and derivatives thereof, refer to the direction generally towards the patient end for use, and the term “proximal” and/or “rearwards” or “rearwardly”, and derivatives thereof, is used to describe the direction away from the patient during use.

As shown in the figures and as will be described, the proximal end of the safety needle device 110 is attachable to a distal end of a medical injector device 112. The distal end of the safety needle device 110 will be pressed against the skin of patient during an injection and the distal end of the medical injector device locates away from the patient.

Such safety needle devices 110 include a spring or a number of springs to urge a shield member and/or to move the needle 116 for safety reasons to provide needlestick protection. In particular, a shield 114 may slide back to expose a patient end of the needle 116 during the injection when the shield is pressed against the skin of a patient. The (inner) shield 114 may then move to a shielding position when removed from the patients' skin which prevents access to the patient (distal) end of the needle 116 after the injection.

Alternatively, or in addition, the safety needle device 110 may be in the form of a spring activated safety needle device 110 which also includes a spring (not shown) to urge and subsequently move a needle mount 140 upon detachment of the spring activated safety needle device 110 from the medical injector device 112. This spring specifically moves the non-patient end 118 of the needle 116 to a shielding position whereby the non-patient end 118 of the needle 116 locates adjacent to or abuts a wall of a tubular housing 130.

The present invention may be used with pen injectors and will be described by way of example as being used with a pen injector. Pen injectors generally include a medical injector device 112 including a dose-adjustment mechanism for setting a dose, for example of insulin, and a safety needle device 110 for insertion into a patient to allow proper drug administration. The pen comprises a single use safety needle and is removed and disposed of after each administered dose.

The pen needle is a double ended needle 116 and includes a patient end 119 or distal end comprising a sharp tip for insertion into a patient. The double ended needle 116 also includes a non-patient end 118 or proximal end comprising a sharp tip for insertion into a drug vial or cartridge provided by the pen injector 112. The proximal/non-patient end 118 of the needle 116 will typically have to pierce a rubber or elastomeric seal 111 which may comprise a septum or stopper provided on the end of the vial or cartridge to access the liquid medicament or drug contained within the cartridge. Spring activated safety needle devices 110 have been developed to shield the distal, or patient end 119 of the needle 116 to prevent an inadvertent “needlestick injury” after use. In addition, spring activated safety needle devices 110 have been developed to shield the proximal, or non-patient end 118 of the needle 116 after detachment from the medical injector device 112 to prevent an inadvertent “needlestick” after use.

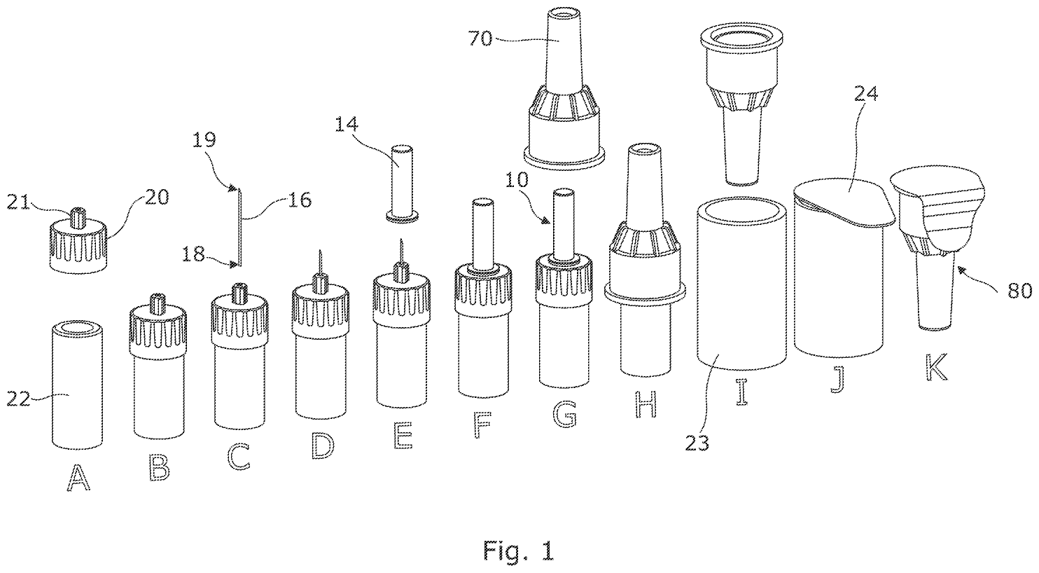

With reference to FIG. 1, a typical standard pen needle assembly line shows the assembly of a standard pen needle 16 including a shield 14 (an inner shield) to shield the distal, or patient end 19 of the needle 16.

Initially, a hub 20 is oriented and placed on an assembly fixture 22. The hub 20 includes a needle mount upstand 21 and this may be prepared for gluing using plasma jets to improve the bonding power of the adhesive. The needle 16 is then oriented with the patient end 19 of the needle 16 pointing upwardly. The patient end 19 of the needle 16 is sharpened to have typically three bevels whereas the non-patient end 18 is sharpened to have typically a single bevel. The needle 16 is then glued and the glue is then cure hardened with heat or ultra-violet light into the upstanding needle mount 21 of the hub 20.

Both the distal and proximal ends of the needle 16 are then silicone coated to lubricate the needle 16 and the needle 16 is air pressure/flow checked to ensure that the needle 16 has no blockages. The needle 16 and the hub 20 combination are video inspected to ensure that the needle 16 is positioned correctly and is located perpendicularly relative to the needle mount 21 and the hub 20 and to ensure no damage has occurred to the needle end. Following this, a shield 14 is oriented and placed carefully over the needle 16 and is secured to the upstand 21 of the needle mount 20 using a press fit frictional arrangement. Again, a video inspection of the shield 14 and needle 16 may be undertaken. This may require the camera to view through the shield 14 to again ensure that the needle 16 was not damaged in any way during placement of the shield 14 over the needle 16.

An open-ended container 70 is oriented and then placed over the shield 14 and the hub 20. The open-ended container 70 is secured with a press fit frictional arrangement around the outer periphery of the hub 20. The completed assembly is then transferred and positioned into a further assembly fixture 23. In this position, a peel-off tamper evident barrier label 24 is sealed over the end face of the open-ended container 70, the label having already been printed with an expiry date corelating to the Use By date for packaged product. The label 24 may be folded down and heat sealed or glued/adhered to the side of the open-ended container 70. Finally, the completed pen needle packs 80 are packaged, typically in boxes of 100, and sterilized using gamma radiation or with ethylene oxide gas. High temperature dry heat can also be sued as a sterilisation process.

As indicated above, the assembly of a simple pen needle involves numerous steps and a number of verification checks may be required after each step or at least specific step involving the insertion/placement of the needle. Accordingly, the assembly process is complex and this may restrict the operational speed.

In particular, the present invention relates to an improved assembly method for spring activated safety needle devices 110. Such devices 110 are more complex than a simple pen needle 10 as described above due to the further complication of inserting and assembling intricate plastic parts and positioning and tensioning spring(s) within the device 110 whilst, critically, avoiding damage to the needle 116 and other components during the assembly.

The present invention provides a method whereby a sub-assembly 101 is initially assembled and this sub-assembly 101 is then introduced into an assembly line 100 thereby simplifying and condensing the assembly line and method. The sub-assembly 101 includes an open-ended container 170, a tubular housing 130, a shield 114, a spring (not shown) and mounting means for a needle mount 140.

Briefly, the present invention provides an assembly line 100 whereby a needle 116 is secured within the needle mount 140. The needle mount 140 is then mounted to the prepared sub-assembly 101 in a single step. Finally, as with the prior art simple pen needle assembly line, a label 124 can be sealed over the open face of the open-ended container 170. Overall, a pre-assembled sub-assembly 101 simplifies the process significantly such that the assembly line 100 requires less inspections which increases the reliability and speed of the process. The needle mount 140 provides a push fit arrangement whereby the needle mount 140 is correctly positioned within the mounting means in a single action. In addition, the tensioning of the spring is automated by the positional arrangement of the needle mount 140 within the tubular housing 130. The mounting means provides a first relative positional relationship between the needle mount 140 and the spring 150 and a second positional relationship between the needle mount 140 and the spring 150.

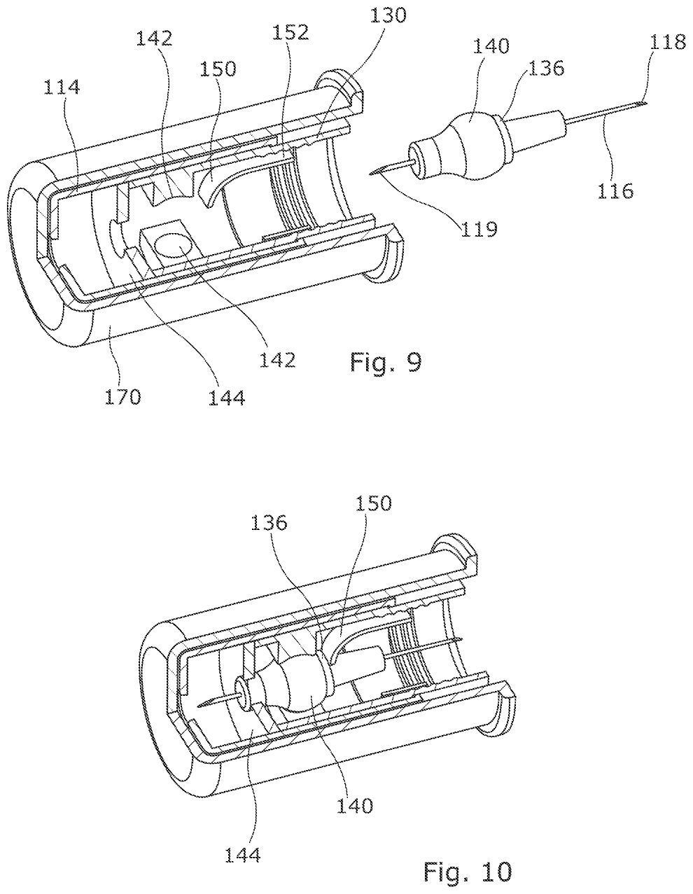

A preferred embodiment of a spring activated safety needle device 110 is shown in FIG. 9 to FIG. 12. The needle mount 140 is positioned within the tubular housing 130 in a fixed position and the spring 150 is located on an inner sleeve section 152 which is movable within the tubular housing 130.

Prior to insertion of the needle mount 140 (see FIG. 9), it will be readily appreciated that the inner sleeve section 152 is held in position relative to tubular housing 130 in order to maintain the inner sleeve section 152 within the tubular housing 130. Any suitable arrangement may be used to maintain this positional arrangement, for example frictional resistance between the inner sleeve section 152 and the tubular housing 130, a latch mechanism, reciprocal projections/recesses, detents etc. on the inner sleeve section 152 and the tubular housing 130 etc. The tubular housing provides two axially opposed recesses 142 (mounting members) which retain the needle mount 140 in a fixed longitudinal position but enables the needle mount 140 to articulate within the mounting members 142. This mounting arrangement provides a ball and socket type joint. With the first sub-assembly coupled/united to the second sub-assembly (as shown in FIG. 10) position, the needle is held along the central longitudinal axis by a blocking member 144.

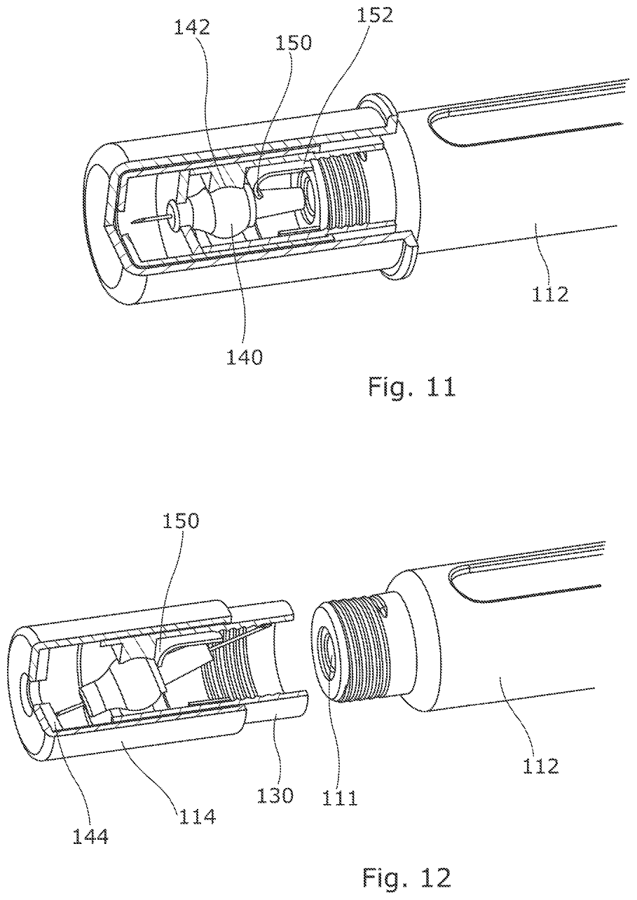

The attachment of the safety needle device 110 to the medical injector 112 causes the spring 150 (mounted on the inner sleeve section 152) to move distally relative to the needle mount 140 in order to load the spring 150, as shown in FIG. 11. Specifically, the attachment of the safety needle device 110 to the medical injector 112 causes the spring 150 and needle mount 140 to shift from the set position (to an active position). In particular, a proximal end of the inner sleeve section 152 abuts and makes contact with a distal end of the medical injector 112 when partially attached. The inner sleeve section 152 is then prevented from moving any further towards the medical injector 112 but the further attachment moves the tubular housing 130 towards the medical injector 112. Accordingly, the tip of the spring 150 moves towards and abuts the flange 136 and the spring 150 is loaded generating a rotational force on the needle mount 140.

Following the attachment of the safety needle device to the medical injector and the subsequent loading of the spring 150, the injection is performed and the shield 114 (outer shield) moves or is moved distally back to a shielding position. During the injection procedure, the blocking means 144 (control member) moves distally and remains at the distal end of the shield 114. In this position, the blocking means 144 no longer maintains the needle 116 along the central longitudinal axis. In this position, the spring 150 is urging the needle mount 140 to rotate but this is counteracted by the engagement of the non-patient end 118 with the rubber seal 111.

The subsequent detachment of the safety needle device 110 from the medical injector 112 releases this counteraction engagement, as shown in FIG. 12. In particular, the non-patient end 118 is released from the rubber seal 111 and the needle mount 140 immediately rotates due to the loading of the spring 150. The needle 116 moves away from an alignment with the central longitudinal axis and the non-patient end 118 is withdrawn into the tubular housing 130 and locates/abuts the internal surface of the tubular housing 130 in a safe condition. It will be readily appreciated that the inner sleeve section 152 is held in position relative to the needle mount 140 in order to maintain the urging force exerted by the spring on the flange 136. Any suitable arrangement may be used to maintain the inner sleeve section 152 in position, for example frictional resistance between the inner sleeve section 152 and the tubular housing 130, a latch mechanism, reciprocal complementary projections/recesses/detents or cam profiles on the inner sleeve section 152 and the tubular housing 130 etc. By way of confirmation, the shift from the set position (to an active position) is permanent to ensure movement of the needle 116 to the shielding position when the safety needle device 110 is detached from the medical injector 112. Therefore, frictional contact (as shown in the drawings) or some other means would be required to hold and maintain the inner sleeve section 152 within the tubular housing 130 at an active position after use.

In alternative embodiments, the needle mount 140 is initially mounted (with the spring 150 not in tension) and the action of securing the safety needle device 110 to a medical injector 112 may move the needle mount 140 (rather than the inner sleeve 152) to a second position whereby the necessary spring force is created. This force creates the urging action to subsequently tilt/pivot the needle 116 as the safety needle device 110 is detached from the medical injector 112 and this provides passive (automatic) needlestick protection of the non-patient end 118 of the needle 116. In other embodiments, the spring 150 may be preloaded as the first sub-assembly 101 is united/coupled to the second sub-assembly 102. However, such embodiments may suffer from degradation during storage and prior to use if a plastic spring is used. For example, the spring 150 may be tensioned as the needle mount 140 is located within the mounting means. The method may comprise mounting the needle mount to form a tiltable arrangement as shown in any of GB2104342.7, GB 2108399.3 and GB 2201934.3.

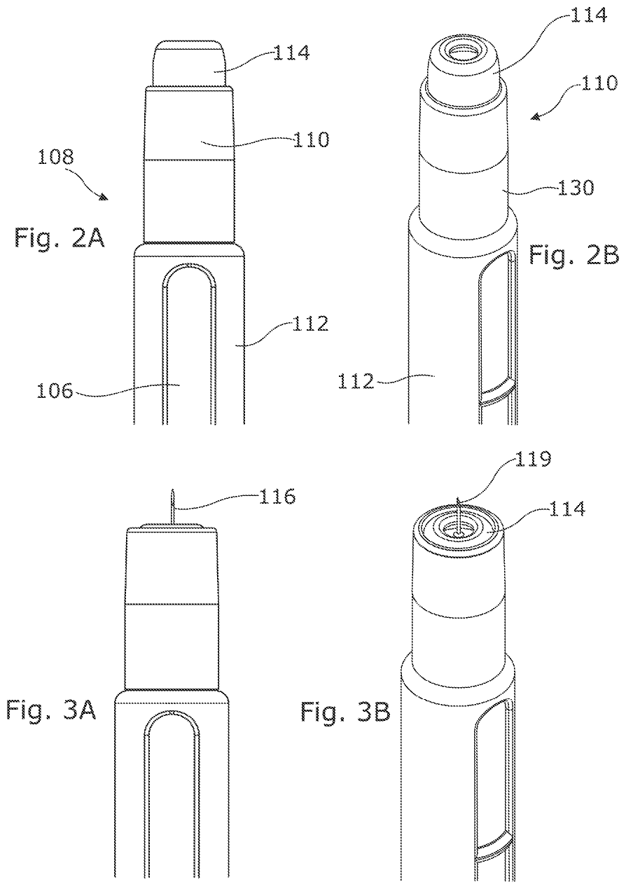

A safety pen needle assembly 110 and a medical injector device 112 are shown in FIGS. 2A and 2B to FIG. 5 and provide an injector 108 in the form of a medical pen or pen injector. The medical injector device 112 comprises a cartridge 106 containing a medicament/fluid and, in particular, the cartridge 106 contains a volume of liquid medicament. The spring activated safety needle device 110 includes a hub or tubular housing 130, a needle 116 and a shielding sleeve 114. The tubular housing 130 includes a body 131 having a distal end 132 and a proximal end 134.

The proximal end 134 is formed with an open face open and is shaped to receive a portion of the injector device 112 to allow the attachment of the spring activated safety needle device 110 to the injector device 112. The spring activated safety needle device 110 may be mounted to the injector device 112 using threads or a surface configuration, such as a tapered surface for a luer-type mounting, or both. The needle 116 may be of any needle design, particularly of any pen needle design. For the description of the present invention, the term needle 116 will be used although this could be replaced by the term cannula which may more specifically refer to the double ended needle 116 of the safety needle device 10. The needle 116 includes a distal end 118 formed for insertion into a patient, and a proximal end 119. The needle 116 is mounted within a needle mount 140 and may be fixed therein using any known technique, such as being adherently fixed to the needle mount 140.

The spring activated safety needle device 110 provides passive needlestick protection (which requires no intervention by the user) to a non-patient end 118 of the needle 116. In particular, the device 110 provides a mechanism whereby the non-patient end 118 of the needle 116 is moved automatically to a shielding position after use. Specifically, the needle mount 140 is movably (pivotably/tiltably) retained in the tubular housing 130 to move from an axially aligned position (relative to the longitudinal axis of the tubular housing 130) to an oblique angle relative to the longitudinal axis of the tubular housing 130 such that the non-patient end 118 of the needle 116 locates adjacent to or abuts an interior wall of the tubular housing 130. In some embodiments, this safety titling movement occurs as the safety needle device 110 is detached for the medical injector device 112. Briefly, the needle 116 is held along the longitudinal axis by an initial retaining mechanism. As the safety needle device 110 is attached to the medical injector device 112 the non-patient proximal end 118 of the needle 116 penetrates through a seal 111 which thereby also holds and maintains the needle 116 along the longitudinal axis. The initial retaining mechanism is (automatically) released during use prior to the safety needle device 110 being detached from the pen injector. Accordingly, during the detachment, the needle 116 is withdrawn from the seal 111 and the urging means moves the non-patient end 118 of the needle 116 to the shielding position.

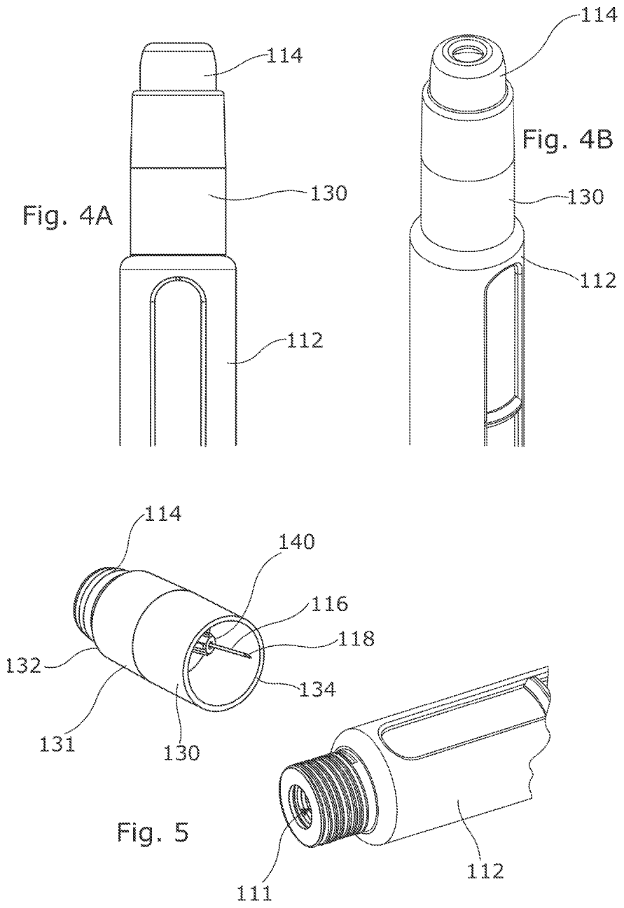

The operational sequence for the spring activated safety needle device 110 is shown in FIG. 2 to FIG. 5. In FIG. 2, the needle device 110 is shown attached to the medical injector device 112 and a front shielding member 114 is in a forward (distal) position which thereby shields the patient end 119 of the needle 116. During an injection (as shown in FIG. 3) the skin of the patient (not shown) causes the front shielding member 114 to retract and the patient end 119 of the needle 116 will penetrate to a desired depth for the injection. As the injector device 108 is removed from the patient, the front shielding member 114 again moves forward to shield the front distal end (tip) 119 of the needle 116, as shown in FIG. 4. Finally, the safety needle device 110 is detached from the medical injector device 112, as shown in FIG. 5. FIG. 5 shows that the needle 116 is titled in order to provide passive needlestick protection to the non-patient end 118 of the needle 116. As shown in FIG. 5, the non-patient end 118 of the needle 116 locates within the tubular housing 130 and at a position adjacent to or abutting the interior wall of the tubular housing 130.

The safety needle device 110 requires an urging means in the form of a spring (not shown) in order to specifically move the non-patient end 118 of the needle 116 to this shielding position. The positioning and mounting arrangement which is needed to provide such movement for these small devices is intricate and requires accurate assembly. Accordingly, this creates problems due to the extra time, cost and steps required in the assembly process. As mentioned previously, the assembly lines and processes for safety needle devices are fast and require numerous verification checks in-between assembly steps. The present invention seeks to address such problems and provides a process involving the formation of two sub-assemblies which are then united to create a needle safety apparatus which in turn significantly speeds up to the process and minimises the number of separate verification steps.

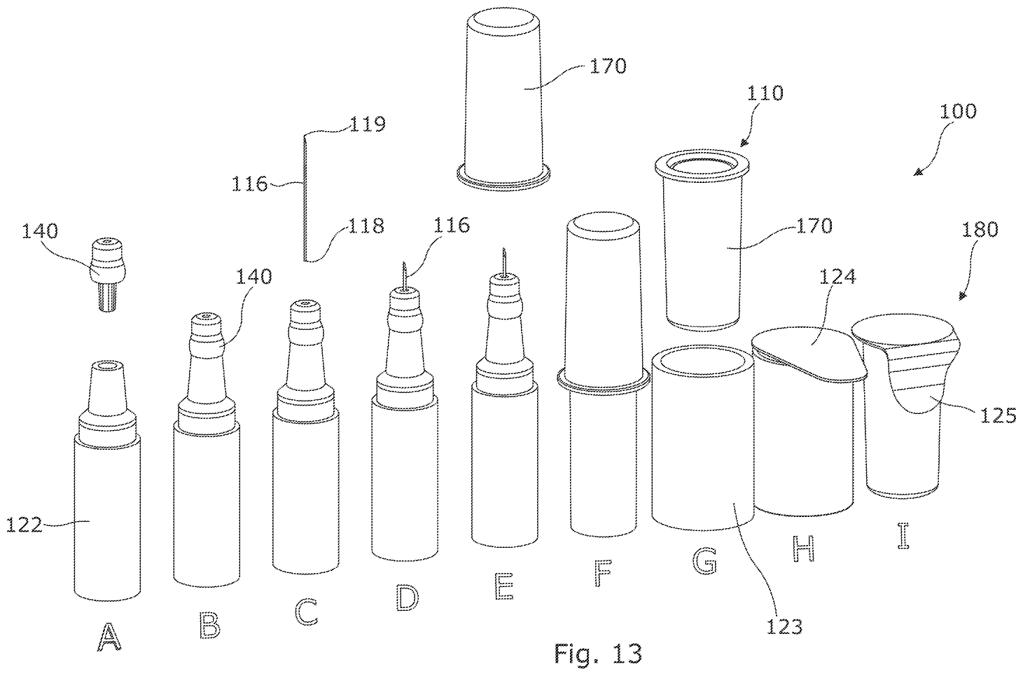

FIG. 13 shows a preferred embodiment of an assembly line 100 (the second assembly line) showing the packaging of a spring activated needle device 110 within a sealed container wherein a first sub-assembly is provided and is introduced into the assembly line. The spring activated needle device 110 includes a shield 114 to shield the distal, or patient end 119 of the needle 116 and a tilting mechanism to provide passive needlestick protection to a non-patient end 118 of the needle 116.

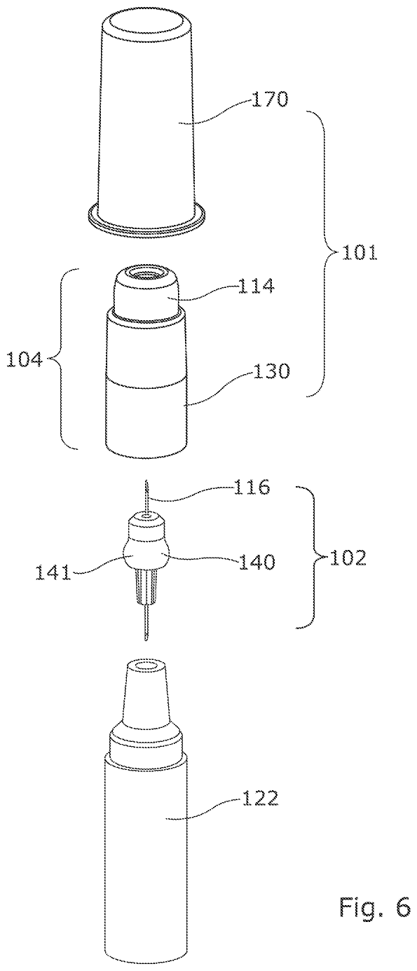

In a first process or first assembly line (not shown), a first sub-assembly 101 is formed which consists of the tubular housing 130, the front shielding member 114 and an open-ended container 170. The combination of the tubular housing 130 and the shielding member 114 forms a telescopic device 104 and this can be assembled and simply inserted into the container 170 in a simple process, as depicted in FIG. 6. Such an assembly is straightforward and can be achieved at speed through a push fit/interference fit type arrangement. For example, an outer surface of the telescopic device 104 being engaged with an inner surface of the container 170. In some embodiments (see FIG. 2-6), the tubular housing 130 has a greater circumference than the inner shield 114 and therefore the tubular housing 130 engages with the inner surface of the container 170 to engage the telescopic device 104 in the container 170. In other embodiments (see FIGS. 9-12) with an outer shield 114, the shield 114 has a greater outer circumference than the tubular housing 130 and, therefore, the shield 114 engages with the inner surface of the container 170 to retain the telescopic device 104 to the container 170.

Alternatively, the shield 114 may be initially inserted into the container 170 and then the tubular housing 130 may be inserted into the shield 114. In some embodiments, the shield 114 locates and slides over a part of the outer surface of the tubular housing 130 (as shown in FIGS. 9-12) and, in other embodiments, the shield 114 locates partly within the tubular housing 130 (as shown in FIGS. 2-6). The inner surface of the container 170 engages with the telescopic device 104 through engagement with either the tubular housing 130 or the shield 114.

Steps A-D in FIG. 13 are similar to a standard assembly line shown in steps A-D in FIG. 1 and, in the present invention, the aim is to form a second sub-assembly 102 in the initial part of this second assembly line. The needle mount 140 is oriented and placed into an assembly fixture 122. In some embodiments, the needle mount 140 may be prepared or readied for gluing using plasma jets in order to improve the bonding power of the adhesive. The needle 116 is oriented with the patient end 119 of the needle 116 projecting upwardly and the needle 116 is placed into the needle mount 140, specifically through a central opening in the needle mount 140. The non-patient end 118 of the needle is sharpened with a single bevel end whereas the patient end 119 is sharpened with three bevels, for example. As shown in schematic step D of FIG. 9, the needle 116 is glued and ultra violet light or heat is used to cure harden the adhesive into the needle mount 140.

The distal and proximal ends of the needle 116 are then coated in silicone for lubrication purposes. The needle 116 is air pressure/flow checked to ensure that the needle 116 is not blocked. The needle 116 and the needle mount 140 are video inspected to ensure that the needle 116 is positioned perpendicularly in the needle mount 140 and to also ensure that the needle tip is not damaged. The combination of the needle mount 140 and the needle 116 form the second sub-assembly 102 for the present invention.

As mentioned above, from the first separate/parallel process, a first sub-assembly 101 is formed in a first separate/parallel assembly line and the first sub-assembly consists of the tubular housing 130 and a shielding member 114 and locating these within an open-ended container 170. The combination of the tubular housing 130 and the shielding member 114 forms a telescopic device 104 and this can be simply inserted into the container 170 in a simple process. Such an assembly is straightforward and can be achieved at speed through a push fit/interference fit or latching type of arrangement. For example, an outer surface of the telescopic device 104 being engaged with an inner surface of the container 170. The first sub-assembly 101 may be formed at a remote location from the second assembly line and in a separate remote facility. At the end of the first assembly line, the first sub-assemblies may be bulk packaged and then transported to the location of the second assembly line or the first (1^{st}) and second (2^{nd}) sub-assembly process takes place at the same location. The first sub-assemblies may then be unpackaged and inserted into the second assembly line as shown in the schematic at step E in FIG. 13. Such packaged sterilised medical products typically have a 5-year Use By shelf life correlating to the date that the product was packaged and sterilised. Each peel-off closure label is date printed during the second assembly line process. Accordingly, it is advantageous to only introduce the first sub-assembly into the second assembly line as and when required (just in time) since the products coming off the second assembly line are destined for destruction in 5 years' time if not purchased and used in the intervening period.

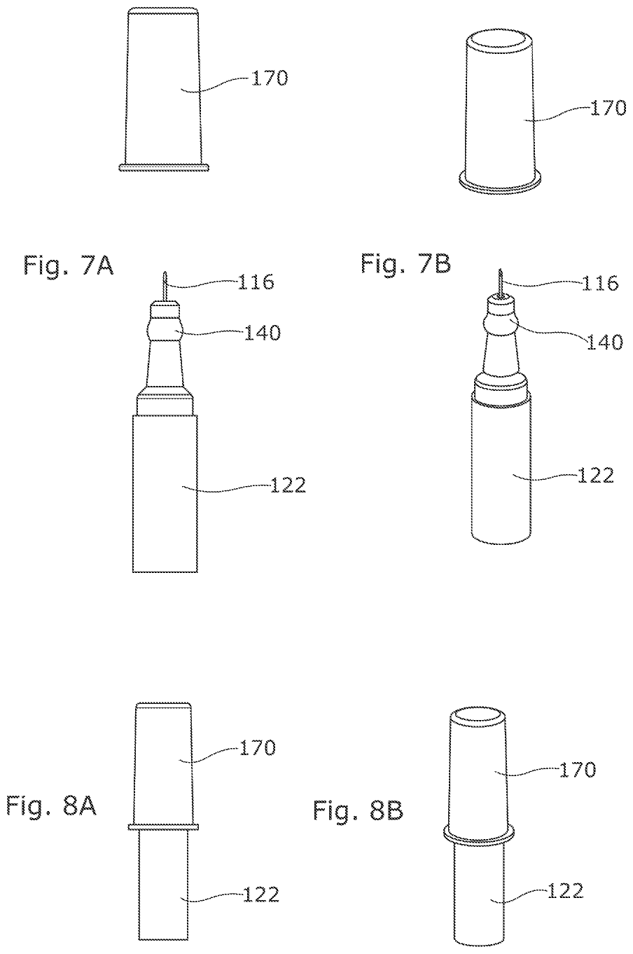

The first sub-assembly 101 is oriented in relation to the second sub-assembly 102 as shown in FIG. 7A and FIG. 7B in schematic step E of FIG. 13 and in FIG. 6 (in an exploded view). The tubular housing 130 provides the spring to create the tilting movement for the passive needlestick protection of the non-patient end 118 of the needle 116. The spring may be an integral part of the tubular housing 130 and may comprise a resilient finger. This spring is in an unloaded configuration in the first sub-assembly 101 since the needle mount 140 (of sub-assembly 102) does not form a part of the first sub-assembly 101. In some preferred embodiments, the spring is provided by a leaf spring/resilient member which projects from within the tubular housing 130. The tubular housing 130 provides one or more mounting elements on an inner surface which are arranged to engage one or more mounting elements provided on the needle mount 140.

In the preferred embodiment, the needle mount 140 comprises a shaped outer surface 141 (mounting element) which is engageable within a shaped inner surface (mounting member) provided by the tubular housing 130. In particular, the shaped outer surface and the shaped inner surface form a ball and socket joint such that the needle mount 140 is axially mounted within the tubular housing. Specifically, the needle 116 is tiltably/pivotably mounted within the tubular housing 130 and is movable from an operative position to a shielding position whereat the needle 116 extends at an angle oblique to a longitudinal axis of the tubular housing 130 to place the non-patient end 118 of the needle 116 at a location adjacent to an interior wall of the tubular housing 130. The needle 116 is initially held by blocking means (not shown) at the operative position whereat the needle 116 extends in a direction along a longitudinal axis of the tubular housing 130 and whereby release of the blocking means allows movement of the needle 116 to the shielding position.

In the preferred embodiment, the shaped outer surface 141 providing the mounting element provides a uniform shaped surface such that the needle mount 140 can be inserted into the tubular housing 130 at any rotational radial position about a central longitudinal axis. For example, the second sub-assembly 102 forming a combination of needle 116 and mount 140 has a central longitudinal axis and the tubular housing 130 of the first sub-assembly 101 also has a central longitudinal axis. Initially, the first sub-assembly 101 is oriented such that the central longitudinal axis is coincident with the central longitudinal axis of the second sub-assembly 102 needle 116 and mount 140. The second sub-assembly 102 can then be simply inserted into the first sub-assembly 101 solely by a relative linear movement in a direction along these coincident longitudinal axes. Specifically, neither the first sub-assembly 101 or the second sub-assembly 102 need to be twisted/rotated about the central longitudinal axis to an exact radial position since the mounting element/mounting member do not require a fixed relative rotational position as would be the case with some mounting arrangements. Accordingly, the ball and socket attachment provided by some preferred embodiments of the two sub-assemblies simplifies the assembly process.

In some embodiments however, the tubular housing 130 provides two axial recesses for receiving two axle stubs projecting outwardly form the needle mount 140. When the axle stubs are engaged within the axial recesses the needle mount 140, the needle mount 140 is in the active position within the safety needle device 110. In some embodiments, the axial stubs can be offset from the longitudinal axis of the needle mount 140.

In some embodiments of the safety needle device 110, the tubular housing 130 is provided with a single pair of mounting elements in order to maintain the needle mount 140 within a fixed axis relative to the tubular housing 130. In particular, the axle mounts for the tubular housing 130 form a single fixed axis about which the needle mount 140 is pivotable or tiltable. However, in some other embodiments, the tubular housing 130 may provide two pairs of mounting elements whereby the needle mount 140 is initially engaged within a first pair of mounting elements. Whilst engaged within the first pair of mounting elements, the spring is maintained in an unloaded position. This may help to maintain the resilient qualities and prevent and deterioration of the safety needle device 110 prior to use. The needle mount 140 is arranged to move to the second pair of mounting elements during use. For example, as the safety needle device 110 is attached to the medical injector device 112, the needle mount 140 is moved from the first pair of mounting elements to the second pair of mounting elements which thereby loads the spring and creates the urging force to subsequently pivot or tilt the needle 116 to the shielding position.

As it will be appreciated, locating and orientating metal compression springs at speed into a number of complex plastic components can easily damage the either end of the needle and this is even more likely when manufacturing safety needle assemblies 110 having back end (non-patient end) needlestick protection whereby a small spring element is arranged to move the needle to a needle shielding position after use. Currently, for this reason, safety needle assemblies 110 including a spring element are assembled in a single assembly line with component parts being introduced sequentially one at a time and with intervening processes and verification steps. For example, as with prior art safety needle assemblies including a spring for a front shielding member for the patient-end of the needle, it would be usual for an unloaded helical coil spring to be carefully located around a delicate needle point during manufacture. The threading of the needle through the central opening of the helical spring is intricate and provides a high risk of damage to the needle. In addition, some designs require the torsional preload winding of the spring to enable the safety device to function, which further complicates the assembly process of these types of devices. A verification step would be needed after this step prior to the securement of the actual shielding member and loading of the spring. The present invention provides for the formation of a first sub-assembly 101 which contains the spring in a static position within the larger first sub-assembly 101. If the safety needle device 110 has a front shielding member 114 then the respective spring for this will already be in a loaded configuration and held statically within the larger open-ended container 170. The first sub-assembly 101 also provides the spring element for the back end protection which, in the preferred embodiments, is not a helical spring and, therefore, the needle 116 does not require the delicate step of being threaded through the central passageway of a helical spring. Overall, the formation of the first sub-assembly 101, creates a large and relatively robust assembly 101 for the easy insertion of the second sub-assembly 102 including the delicate needle 116. Once joined or coupled together, the complete safety needle device 110 is created and the final packaging stage can be completed.

As shown in FIG. 8 and in step F of FIG. 9, the second sub-assembly 102 is located and secured within the first sub-assembly 101. As previously described, this coupling action correctly positions the spring for the back end needlestick protection arrangement since the tubular housing 130 provides a fixed mounting position for the second sub-assembly 102 whereby the spring is correctly positioned relative to the needle mount 140. Specifically, the open-ended container 170, containing the other components of the first sub-assembly 101, is oriented and placed over the needle mount 140 and is press-fitted down to locate over the needle mount 140 in the operative position for the tiltable safety needle device 110. Video inspection can be used to now look through the transparent open-ended container 170 to ensure that the needle 116 was not damaged during fitting of the two sub-assemblies 101, 102 together.

In this configuration the safety needle device 110 is complete and self-supporting but is retained within the open-ended container 170 by an interference fit or a similar arrangement. Such an arrangement enables the safety needle device 110 to be easily extracted from the open-ended container 170 ready for use by the end user.

Once the first sub-assembly 101 has been coupled to the second sub-assembly 102, the outer container can be completed. Initially the assembly is inverted and is retained into a second assembly fixture 123, as shown in step G. In this orientation, the open end of the container 170 is facing upwardly. This enables a cover label 124 to be sealed over the open face in order to complete the container. In particular, a peel off Use By date-stamped label 124 is sealed over the open face. The label also provides a form of “tamper evidence” to confirm the sealed container has not been opened.

As indicated in step I of FIG. 9, a portion of the label 124 in this instance has been folded down and adhered to the side of the container 170. In particular, a tab 125 of the label 124 is heated sealed or glued to the side of the container 170.

Finally, the assemblies 180 are packaged (typically in boxes or cartons of 100) and later sterilized using gamma irradiation or ethylene oxide (ETO) gas. High temperature (dry heat) can also be used as a sterilization process.

As mentioned previously, the term “use” or “during use” are used to cover the complete use of the safety needle device, for example from the act of attaching a medical injector to the safety needle device and removing it from the container, then performing the injection on a patient, and then detaching the safety needle device from the medical injector and finally disposing of the safety needle device into a sharps waste container.

The invention claimed is:

1. A method of assembling and packaging a medical needle device having a double ended needle, which comprises a container having a closed end and an open end to receive a spring activated safety needle device to provide passive needlestick protection to a non-patient end of the needle; and wherein the open end of the container receives a closure to maintain sterility within the container, the method comprising:
performing the independent steps of:
(i) assembling a first sub-assembly comprising a telescopic device engaged within the container wherein the telescopic device comprises a shield and a tubular housing, and wherein the tubular housing comprises:
(a) a mounting member on an internal surface to engage a mounting element of a needle mount; and
(b) a spring means to urge movement of the double ended needle during use to a shielding position whereat the needle extends at an angle oblique to a longitudinal axis of the tubular housing to place the non-patient end of the needle at a location adjacent to an interior wall of the tubular housing;
(ii) assembling a second sub-assembly by securing the double ended needle within the needle mount, wherein the mounting element is provided on an external surface of the needle mount;
inserting the second sub-assembly into the tubular housing to secure the mounting element of the needle mount to the mounting member of the tubular housing to unite the second sub-assembly to the first sub-assembly to enable the spring means to urge movement of the double ended needle during use to a shielding position;
applying the closure over the open end of the container to seal the medical needle device within the container.

2. A method of assembling and packaging a medical needle device according to claim 1 in which the assembling of the first sub-assembly is undertaken on a first assembly line and the assembling of the second sub-assembly is undertaken on a second assembly line.

3. A method of assembling and packaging a medical needle device according to claim 2 in which the first sub-assembly is united to the second sub-assembly on the second assembly line.

4. A method of assembling and packaging a medical needle device according to claim 2 in which the first assembly line is separate and distinct from the second assembly line.

5. A method of assembling and packaging a medical needle device according to any one of claim 2 in which the first assembly line is remotely located from the second sub-assembly line and the method comprises transporting the first sub-assemblies from the first assembly line to the second assembly line.

6. A method of assembling and packaging a medical needle device according to claim 5 in which the method comprises packing a plurality of first sub-assemblies into a package, transporting the package to the second assembly line, removing the first sub-assemblies from the package and individually separating the first sub-assemblies form each other and introducing the first sub-assemblies into the second assembly line.

7. A method of assembling and packaging a medical needle device according to claim 1 in which the method comprises push-fitting the second sub-assembly to the first sub-assembly in order to unite the first and second sub-assemblies.

8. A method of assembling and packaging a medical needle device according to claim 1 in which the uniting of the first sub-assembly with second sub-assembly may comprise moving the first sub-assembly along a central longitudinal axis relatively towards the second sub-assembly and the uniting may comprise this single linear movement.

9. A method of assembling and packaging a medical needle device according to claim 1 in which the method comprises aligning the first sub-assembly with the second sub-assembly wherein this alignment only requires an alignment of central longitudinal axes of the first and second sub-assemblies and omits any relative rotational alignment of the sub-assemblies about the respective central longitudinal axis.

10. A method of assembling and packaging a medical needle device according to claim 1 in which the method comprises aligning a central longitudinal axis of the first sub-assembly with a central longitudinal axis of the second sub-assembly and then solely moving the first sub-assembly along the coincident central longitudinal axes relatively towards the second sub-assembly.

11. A method of assembling and packaging a medical needle device according to claim 1 in which the needle mount is rotatable about a central longitudinal axis in order for the needle mount to remain engageable within the mounting member at any relative rotational position.

12. A method of assembling and packaging a medical needle device according to claim 1 in which the method comprises tiltably mounting the needle mount within the tubular housing as the first sub-assembly is united with the second sub-assembly.

13. A method of assembling and packaging a medical needle device according to claim 1 in which the method comprises preloading the spring as the first sub-assembly is united with the second sub-assembly.

14. A method of assembling and packaging a medical needle device according to claim 1 in which the assembling of the first sub-assembly comprises securing the tubular housing in the open-ended container using an interference fit.

15. A method of assembling and packaging a medical needle device according to claim 1 in which the assembling of the first sub-assembly comprises forming the telescopic device and then removably inserting the telescopic device into the container to form the first sub-assembly wherein the method comprises push fitting the telescopic device into the container.

16. A method of assembling and packaging a medical needle device according to claim 1 in which the uniting of the first sub-assembly with the second sub-assembly comprises push fitting the mounting element into the mounting member.

17. A method of assembling and packaging a medical needle device according to claim 1 in which the method comprises inserting the second sub-assembly into the first sub-assembly with the sub-assemblies being in any relative rotational orientation about a central longitudinal axis.

18. A method of assembling and packaging a medical needle device according to claim 1 in which the mounting element comprises a ball-shaped outer surface of the needle mount which facilitates a ball and socket joint together with the mounting member.

19. A method of assembling and packaging a medical needle device according to claim 1 in which the method comprises locating first and second mounting elements within first and second mounting members respectively.

20. A method of assembling and packaging a medical needle device according to claim 1 in which the method comprises mounting the needle in an operative position, and mounting the spring and the needle mount at a set position, thereafter use of the spring activated safety needle device with a medical injector causes a shift from the set position, said shift enables the spring to rotate the needle to the shielding position when the spring activated safety needle device is detached from the medical injector.

21. A method of assembling and packaging a medical needle device according to claim 1 in which the spring means comprises a resilient member which extends inwardly from the tubular housing and contacts an outer surface of the needle mount at a position offset from an axis of rotation and applies a rotational force to rotate the needle mount.

22. A method of assembling and packaging a medical needle device according to claim 1 in which the spring comprises a leaf spring projecting inwardly from the tubular housing and, with the needle in an operative position, the leaf spring is in a preloaded condition and may be deflected from a neutral/relaxed position.

23. A method of assembling and packaging a medical needle device according to claim 1 in which the needle mount comprises a unitary component having an integral mounting element or mounting elements in the form of a shaped surface or axial members located on an outer surface.
